(19) United States
(12) Patent Application Publication
(10) Pub. No.: US 2011/0087323 A1
(43) Pub. Date: Apr. 14, 2011

(54) Apparatus for Mitral Lifting Annuloplaty

(75) Inventor: Meong-Gun Song, Seoul (KR)

(73) Assignee: SCIENCITY CO., LTD., Kangwon-do, KR (KR)

(21) Appl. No.: 12/922,437

(22) PCT Filed: Mar. 14, 2008
(86) PCT No.: PCT/KR08/01442

§ 371 Date: Dec. 13, 2010

(30) Foreign Application Priority Data
Mar. 13, 2008 (KR) 10-2008-0023311

(51) Int. Cl. A61F 2/24

(52) U.S. Cl. 623/2.36

(57) ABSTRACT

Provided is a mitral annuloplasty band having a size that allows it to be inserted into the ventricle and a rectangular parallelepiped shape extending in a longitudinal direction thereof, wherein the ratio of the width and height is in a range of 1:2 to 1:8.

Therefore, a portion of the mitral annulus adjacent to the posterior leaflet can be lifted up to a mitral annuloplasty band located at the wall of the left atrium and fixed thereto to recover the function of the mitral valve, without disturbing movement of the mitral valve and movement of the mitral annulus and the left ventricle adjacent thereto.

### TECHNICAL FIELD

The present invention relates to a mitral annuloplasty band, and more particularly, a mitral annuloplasty band used in mitral annuloplasty of mitral valvular disease such as mitral regurgitation.

### BACKGROUND ART

The heart has four valves for guiding blood flow forward through two sides of the heart. The left side (systemic circulation) of the heart has a mitral valve located between the left atrium and the left ventricle, and an aortic valve located between the left ventricle and the aorta. The two valves guide oxygenated blood discharged from the lungs to the aorta through the left side of the heart to distribute the blood to the body.

The right side (pulmonary circulation) of the heart has a tricuspid valve located between the right atrium and the right ventricle, and a pulmonary valve located between the right ventricle and the pulmonary artery. The two valves guide the deoxygenated blood coming from the body to the pulmonary artery through the right side of the heart to distribute the blood to the lungs, where the blood is oxygenated before it circulated again.

The four valves may be referred to as a passive structure because the four valves consume no energy and perform no active contraction. They are formed of movable leaflets designed to open and close in response to differential pressures across the valves.

In particular, the mitral valve and the tricuspid valve, which guide the blood flow forward, are referred to as atrioventricular valves because the valves are located between the atria and the ventricles. The atrioventricular valves have papillary muscles originating from the ventricles and chordae tendineae originating from the papillary muscles spread in an umbrella shape to act as a valve.

Here, the mitral valve located between the left atrium and the left ventricle closes upon contraction of the heart and opens upon expansion.

The mitral valve is located between the left atrium and the left ventricle which are formed of muscles, and a belt-shaped annulus formed of fibers is located at the edge of the mitral valve. Two leaflets referred to as an anterior leaflet and a posterior leaflet are located in the mitral valve, and chordae projecting from the two papillary muscles in the left ventricle are connected to lower parts of the leaflets to prevent the leaflets from being pushed by systolic pressure in the left ventricle and inclined toward the left atrium.

A disease in which the mitral valve cannot be completely opened when the heart expands is referred to as mitral stenosis, and a disease in which the mitral valve cannot be completely closed when the heart contracts is referred to as mitral regurgitation.

Mitral stenosis is generated as a sequela of rheumatism or congenital defects. Mitral regurgitation is generated due to rheumatism, degenerative variations of a valve, ischemic heart disease of the myocardium, external injuries such as bacterial or fungal infection, and so on, so that the leaflet cannot be completely closed, or a subvalvular structure of the valve is deformed.

When the function of the mitral valve is imperfect due to such reasons, blood flow is disturbed, thereby interfering with expansion of the left atrium and the left ventricle, blood flow, and decreasing the contractile force of the heart.

In order to solve these problems, a method of recovering the function of the mitral valve may be an artificial heart valve replacement or mitral annuloplasty.

Artificial heart valve replacement is a widely used method that includes cutting the mitral leaflet through a surgical operation and inserting a mechanical valve or a tissue valve into the incision. However, when a mechanical valve is used, thrombosis may occur, and the patient must constantly take anticoagulants. On the other hand, when a tissue valve is inserted, the usually poor durability of the tissue valve requires reoperation after a certain period. That is, both methods are surgical operations that break the subvalvular structure of the mitral valve and thus decrease the function of the left ventricle.

In order to overcome these problems, mitral annuloplasty is used to recover the valvular function of the mitral valve while maintaining the patient's mitral leaflet and subvalvular structure.

The mitral annuloplasty must prevent bulging of the annulus to recover the function of the mitral valve, the annulus and the left ventricle must move together depending on a contraction period of the ventricle, and the leaflet and the subvalvular structure must perfectly recover the valvular function of the leaflet.

Up to now, various instruments for mitral annuloplasty have been developed to perform the necessary functions of the procedure. For example, the Carpentier-Edwards Classic Mitral annuloplasty ring available from Edwards Lifesciences LLC in France has attempted to recover the function of the annulus using a solid D-shaped forming apparatus. However, as a result of disregarding the biomechanical characteristic that the annulus moves together with the ventricle, the annulus corresponding to an anterior leaflet of the mitral valve is fixed to sequentially disturb closing of the leaflet and subvalvular structure, thereby narrowing the flow to the aorta from the left ventricle. In addition, the Duran Flexible Annuloplasty Ring available from Medtronic Inc. in the USA has been developed as a flexible mitral annuloplasty ring by considering the movement of the annulus. However, the annuloplasty ring and the annulus are adhered to each other due to the function and shape of surrounding the entire annulus and tightening it, thereby reducing the area of the mitral valve.

In order to overcome the problems of the flexible mitral annuloplasty ring developed by Duran, a Cosgrove-Edwards Annuloplasty System available from Edwards Lifesciences LLC in USA has been developed as a flexible mitral annuloplasty apparatus for fixing a part of the annulus corresponding to a posterior leaflet. However, the annuloplasty apparatus and the annulus are also adhered to each other so they act as a solid fixture, thereby limiting movement of the posterior leaflet and peripheral cardiac muscle (see FIGS. 3a and 3b).

Here, FIG. 3a shows a mitral annulus, in particular, a posterior mitral annulus, before a mitral annuloplasty ring is fitted, and FIG. 3b shows the fitting of a conventional mitral annuloplasty ring.

In particular, as shown in FIGS. 2a, 2b and 4, the Duran ring or the Cosgrove ring, both of which are flexible rings, are fixed to the mitral annulus to effectuate only a vector parallelly fixing the mitral annulus to the surface of the annulus. As a result, it is possible to suppress expansion of the ring in a radial vector (Y-direction) perpendicular to a plane in a longitudinal vector (X-direction), but it is impossible to suppress deformation in a vertical vector (Z-direction), for example, contraction. Actually, the mitral annulus has a small variation in height, not located on the same plane (X-Y plane). Therefore, since the Duran ring or the Cosgrove ring is located on the mitral leaflet adjacent to the mitral annulus as shown in FIG. 4, a gap is generated between the ring and the mitral annulus, and then, the gap is filled with granulation tissues as time elapses, thereby limiting movement of the annulus.

As a result, the Duran ring and the Cosgrove ring can receive an X-Y direction force on the X-Y plane to fix the mitral annulus, but cannot resist against the Z-direction deformation. Therefore, the Duran ring and the Cosgrove ring are deformed and shortened by the Z-direction force, and finally, the area of the mitral valve is reduced due to variation in X-Y direction force and length to generate imperfect closing of the mitral leaflet.

### DISCLOSURE

### Technical Problem

The present invention provides a mitral annuloplasty band capable of preventing bulging of a mitral annulus, and recovering the function and subvalvular structure of a mitral leaflet while maintaining the function of the mitral leaflet and the mitral annulus, without disturbing the movements thereof, in consideration of the function of the mitral annulus.

### Technical Solution

According to an exemplary embodiment of the present invention, a mitral annuloplasty band fixes the mitral annulus adjacent to a posterior leaflet without producing bulge of the annulus. On the basis of the fact that the height of the mitral annulus is not on the same plane, the annulus is lifted up and fixed to the height of the apparatus in accordance with the present invention located on the left atrium, not on the mitral valve. Therefore, it is possible to provide a mitral annuloplasty band capable of preventing deformation in a vertical direction (Z direction), and formed of a flexible fiber material.

### DESCRIPTION OF DRAWINGS

The above and other aspects and advantages of the present invention will become apparent and more readily appreciated from the following description of exemplary embodiments, taken in conjunction with the accompanying drawings, in which:

FIG. 1 is a view showing the structure of the heart;

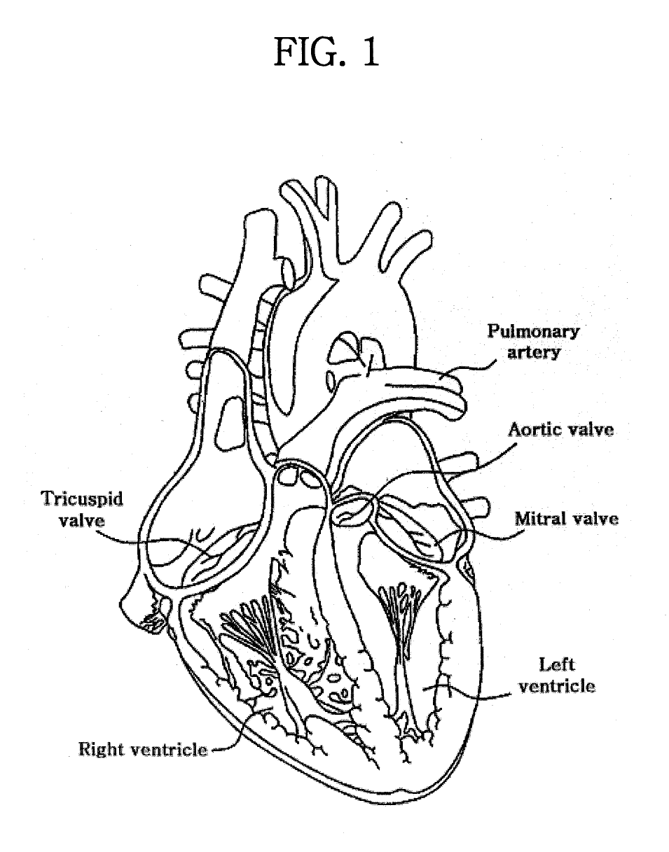

FIG. 2a is a view showing vectors when a mitral annuloplasty ring is in operation;

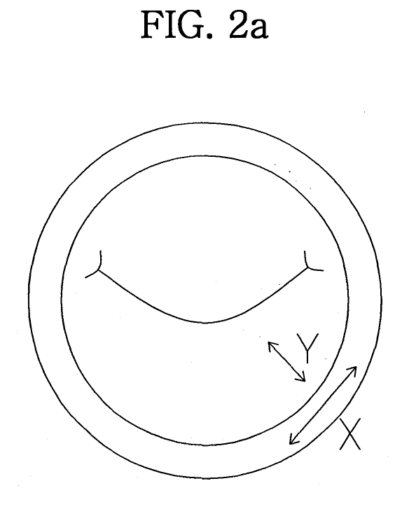

FIG. 2b is a view showing vectors of the mitral annuloplasty ring in imaginary three-dimensional coordinates;

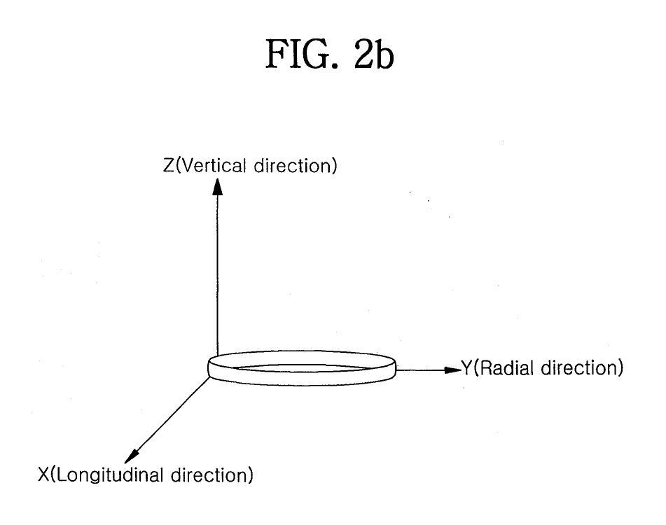

FIG. 3a is a view showing a mitral annulus before the mitral annuloplasty ring is fitted;

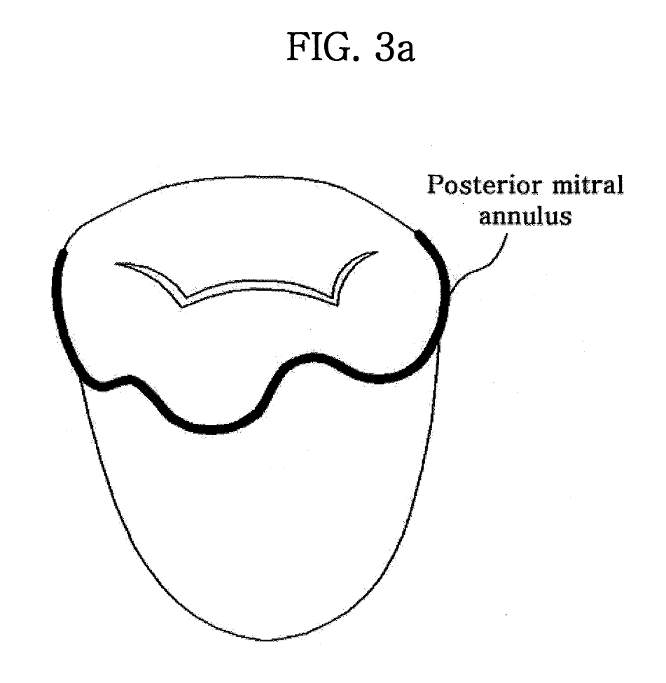

FIG. 3b is a view showing how a conventional mitral annuloplasty ring is fitted;

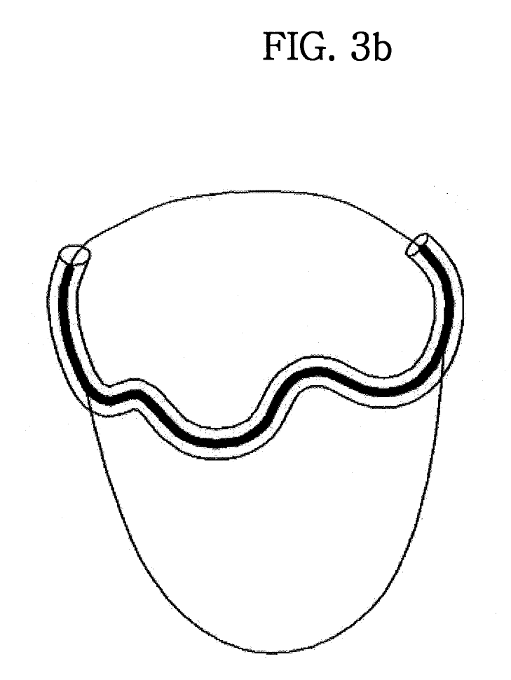

FIG. 4 is a view showing how the conventional mitral annuloplasty ring is located on a leaflet including an annulus;

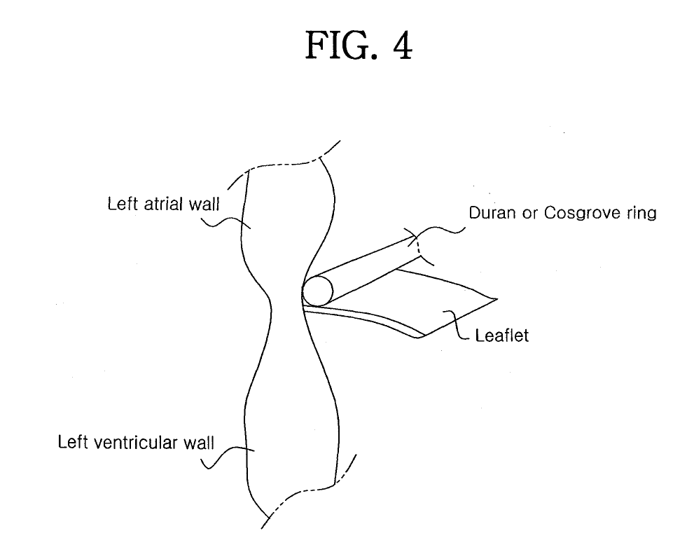

FIG. 5 is a perspective view of a mitral annuloplasty band in accordance with an exemplary embodiment of the present invention;

FIG. 6 is a cross-sectional view of the mitral annuloplasty band in accordance with an exemplary embodiment of the present invention;

FIG. 7 is a view showing vectors of the mitral annuloplasty band in accordance with an exemplary embodiment of the present invention in imaginary three-dimensional coordinates;

FIG. 8 is a view of a mitral valve as seen from the left atrium;

FIG. 9 is a view showing a position where the mitral annuloplasty band is stitched to the mitral annulus with reference to line B-B′ of FIG. 8;

FIG. 10 is a view showing how the mitral annuloplasty band in accordance with an exemplary embodiment of the present invention is stitched to the mitral annulus;

FIG. 11 is a view showing how the mitral annuloplasty band in accordance with an exemplary embodiment of the present invention was stitched to the mitral annulus;

FIG. 12 is a view showing how the mitral annuloplasty band in accordance with an exemplary embodiment of the present invention is fitted to the mirtral annulus;

FIG. 13 is a view showing an adaptation of a suture when the mitral annuloplasty band in accordance with an exemplary embodiment of the present invention is fitted;

FIG. 14 is a view showing the shape of the mitral valve and the left ventricle, with mitral annuluses having varying height with reference to an imaginary plane, before the mitral annuloplasty band is fitted;

FIG. 15 is a view showing a process of lifting a posterior mitral annulus of varying height using the mitral annuloplasty band in accordance with an exemplary embodiment of the present invention;

FIG. 16 is a view showing the shape of the mitral valve and the left ventricle when the mitral annuloplasty band in accordance with an exemplary embodiment of the present invention is fitted; and

FIG. 17 is a plan view showing the shape of the mitral valve as seen from the left atrium when the mitral annuloplasty band in accordance with an exemplary embodiment of the present invention is fitted.

### MODE FOR INVENTION

The present invention provides a mitral annuloplasty band having a size that allows the band to be inserted into the atrium and a rectangular shape extending in a longitudinal direction thereof, with a ratio between the width and height of 1:2 to 1:8.

A guide groove may further be formed in an upper end of one side of the elongated rectangular parallelepiped along a longitudinal direction thereof.

In addition, the mitral annuloplasty band may be transformable in the longitudinal direction, and nontransformable in a vertical direction thereof.

In general, mitral incompetence includes mitral stenosis in which the mitral valve cannot be completely opened and mitral insufficiency in which the mitral valve cannot be completely closed. Causes of the mitral stenosis disturbing the complete opening of the mitral valve upon a contraction period of the heart include a sequela of rheumatic fever, congenital malformation, and so on. Causes of the mitral insufficiency disturbing the complete closing of the mitral valve upon a contraction period of the heart include expansion of the annulus, bulge or injury of the leaflet, breakage of subvalvular structures such as chordae tendineae and papillary muscles, hanging-down thereof, and so on.

A mitral annuloplasty band is used in mitral annuloplasty of mitral diseases such as valvular incompetence of the mitral valve of the heart. The mitral annuloplasty band in accordance with the present invention may include all members capable of lifting up the mitral annulus adjacent to the posterior leaflet to a wall of the left atrium and fixing it thereto to prevent bulge of the annulus, and completely closing the mitral leaflet while maintaining the contraction and expansion functions of the annulus, thereby providing the function as a reference plate.

The present invention will be described more fully hereinafter with reference to the accompanying drawings, in which exemplary embodiments of the invention are shown. This invention may, however, be embodied in different forms and should not be construed as limited to the embodiments set forth herein.

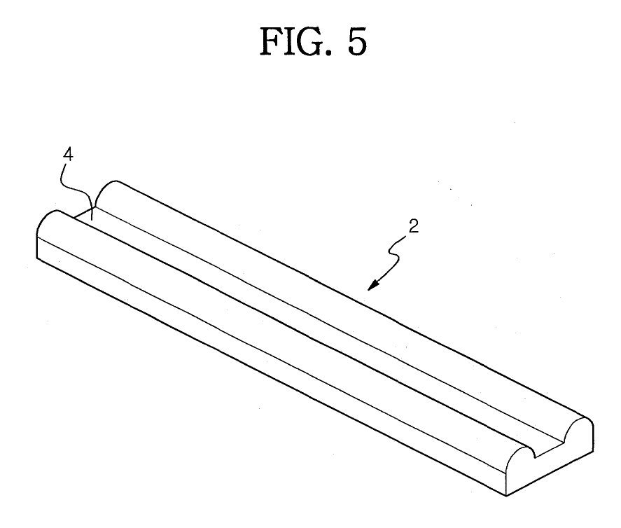

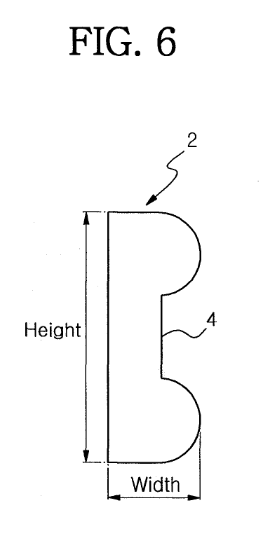

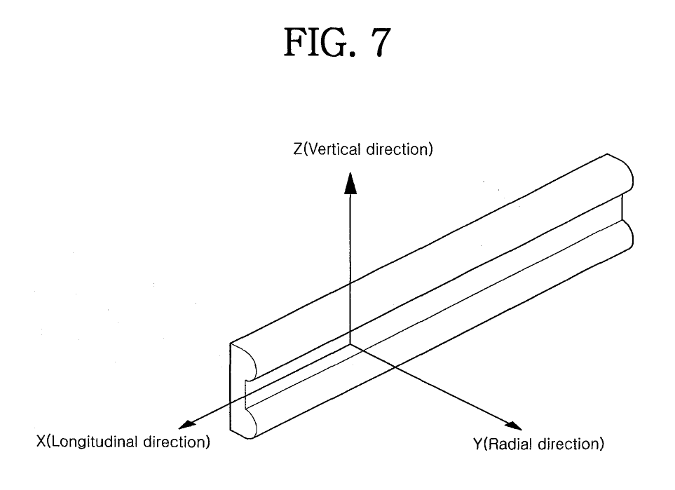

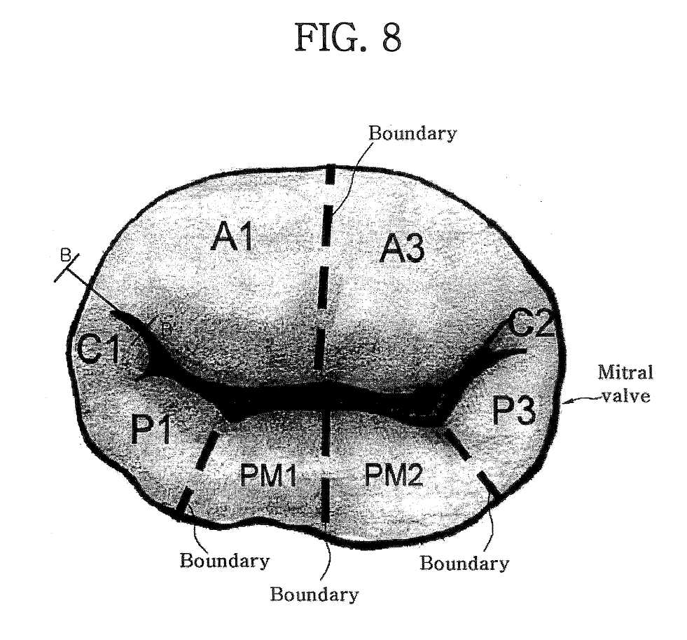

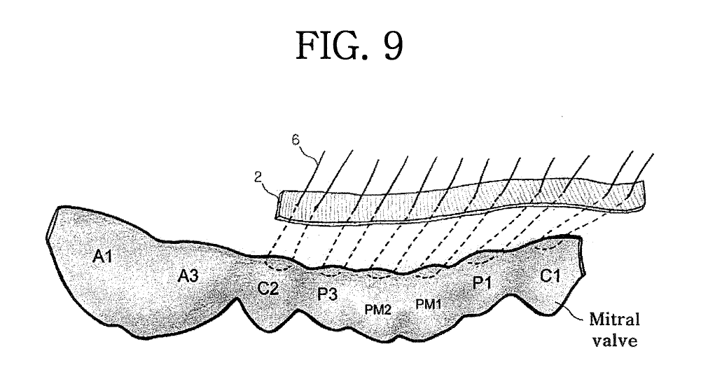

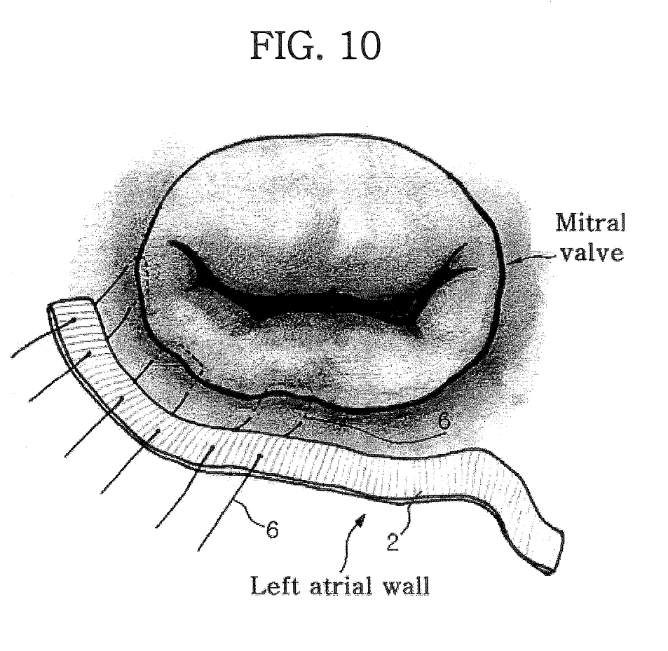

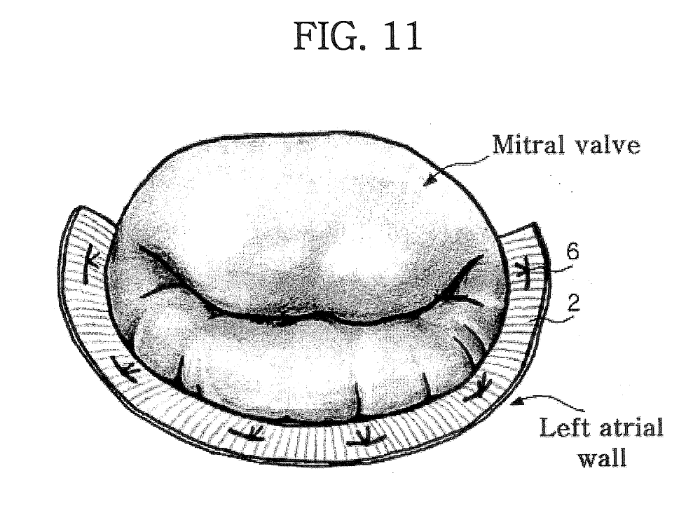

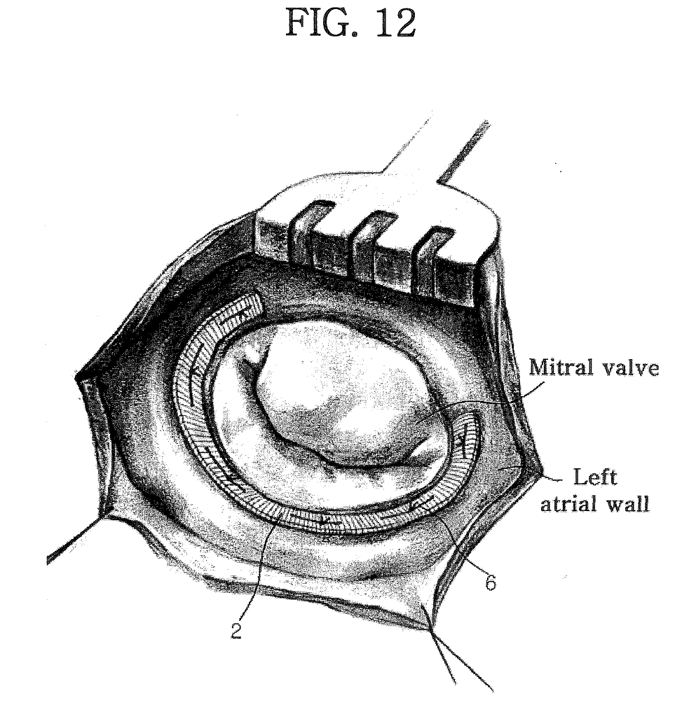

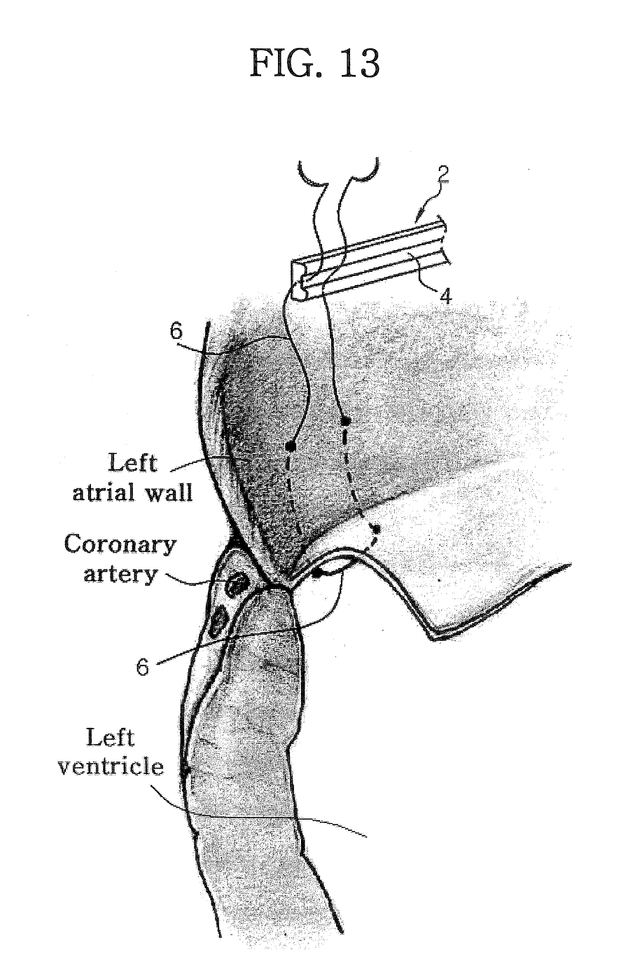

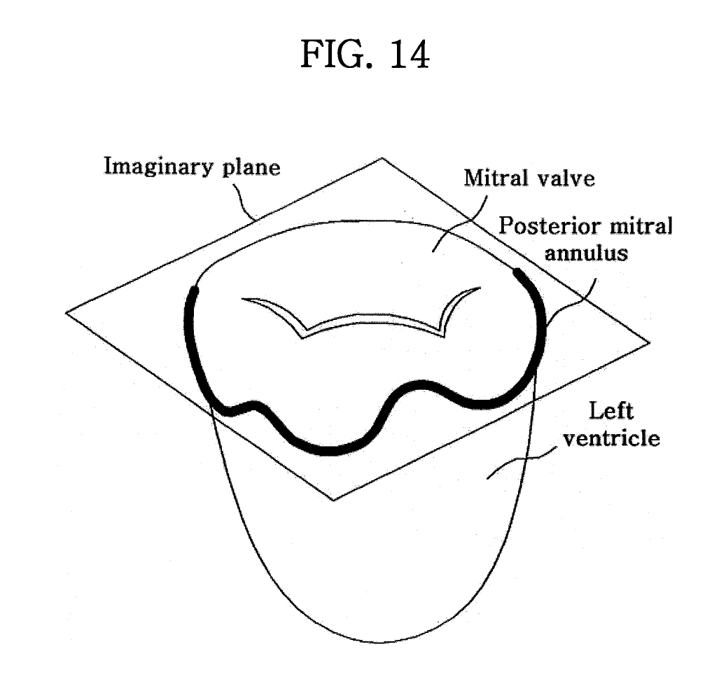

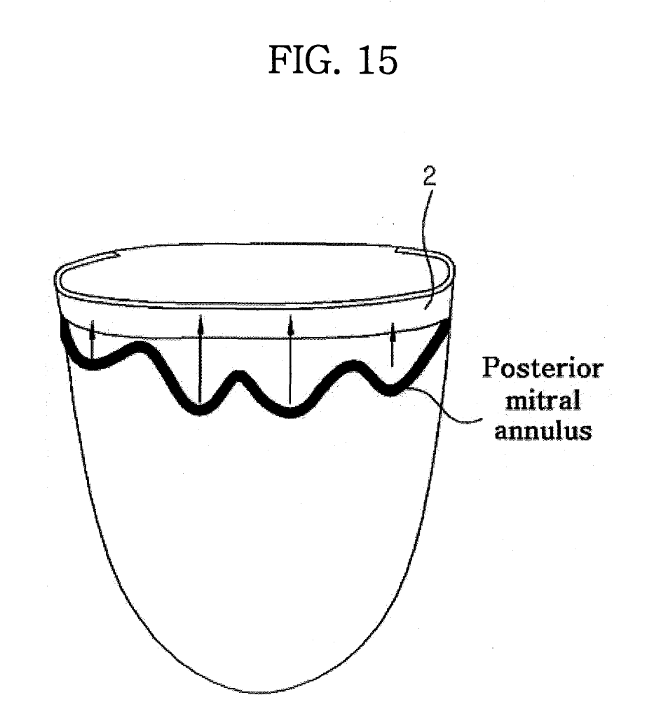

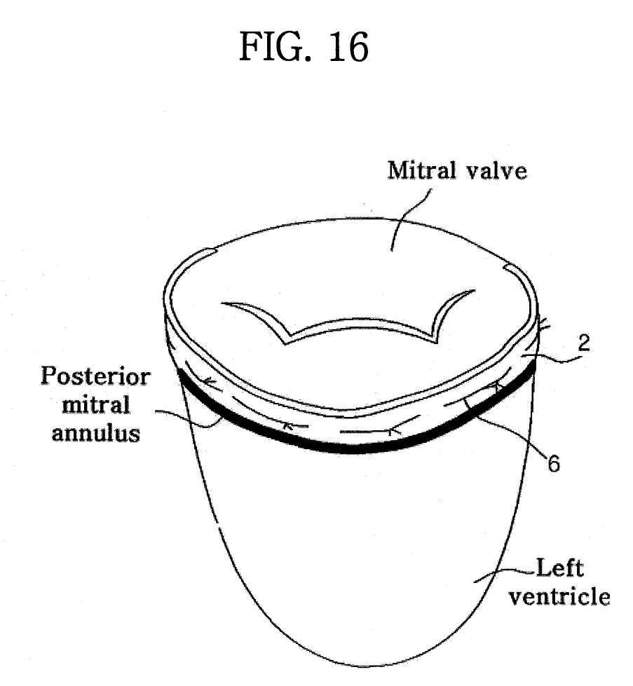

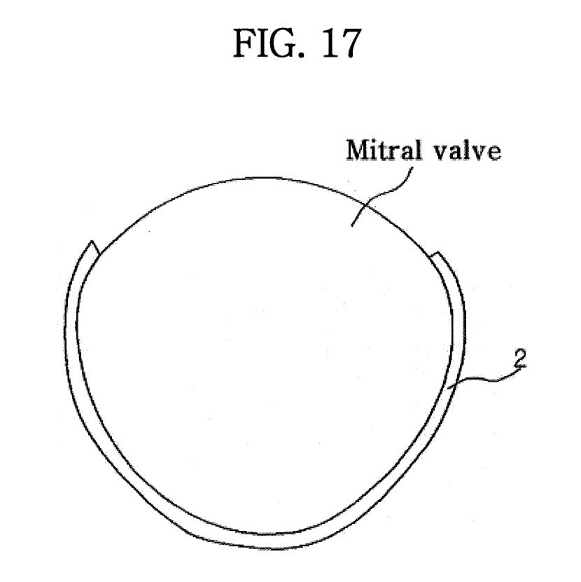

FIG. 5 is a perspective view of a mitral annuloplasty band in accordance with an exemplary embodiment of the present invention, FIG. 6 is a cross-sectional view of the mitral annuloplasty band in accordance with an exemplary embodiment of the present invention, FIG. 7 is a view showing vectors of the mitral annuloplasty band in accordance with an exemplary embodiment of the present invention in imaginary three-dimensional coordinates, FIG. 8 is a view showing a mitral valve as seen from the left atrium, FIG. 9 is a view showing a position where the mitral annuloplasty band is stitched to the mitral annulus with reference to line B-B′ of FIG. 8, FIG. 10 is a view showing how the mitral annuloplasty band in accordance with an exemplary embodiment of the present invention is stitched to the mitral annulus, FIG. 11 is a view showing how the mitral annuloplasty band in accordance with an exemplary embodiment of the present invention was stitched to the mitral annulus, FIG. 12 is a view showing how the mitral annuloplasty band in accordance with an exemplary embodiment of the present invention is adapted to the mirtral annulus, FIG. 13 is a view showing an adaptation of a suture when the mitral annuloplasty band in accordance with an exemplary embodiment of the present invention is adapted, FIG. 14 is a view showing the shape of the mitral valve and the left ventricle with mitral annuluses having varying height with reference to an imaginary plane before the mitral annuloplasty band is fitted, FIG. 15 is a view showing a process of lifting a posterior mitral annulus having a varying height using the mitral annuloplasty band in accordance with an exemplary embodiment of the present invention, FIG. 16 is a view showing the shape of the mitral valve and the left ventricle when the mitral annuloplasty band in accordance with an exemplary embodiment of the present invention is fitted, and FIG. 17 is a plan view showing the shape of the mitral valve as seen from the left atrium when the mitral annuloplasty band in accordance with an exemplary embodiment of the present invention is fitted.

As shown in FIGS. 5 to 17, the mitral annuloplasty band 2 in accordance with the present invention has a geometrically longish shape, e.g., a rectangular shape extending in a longitudinal direction thereof. The mitral annuloplasty band 2 may have any length that allows it to be inserted into the left atrium, preferably, sufficient to surround the mitral annulus in the left atrium. In addition, the mitral annuloplasty band 2 may be formed of any biocompatible material that generates no chemical reactions or side-effects even when the band is inserted into a human body, preferably, polytetrafluoroethylene, polypropylene, nylon, silk, polyurethane, polyester, or a mixture thereof, or a fiber material including, most preferably, polyester as a flexible fiber material.

In particular, the mitral annuloplasty band 2 in accordance with an exemplary embodiment of the present invention has a rectangular shape extending in a longitudinal direction. As shown in FIG. 15, the mitral annuloplasty band 2 is located at a certain height on a sidewall of the left atrium adjacent to the posterior leaflet of the mitral valve to lift up a portion of the annulus adjacent to the posterior leaflet, i.e. an edge of a fiber layer of the posterior annulus, and fix it to the wall of the left ventricle, thereby preventing further bulge of the diameter of the mitral annulus from its original diameter.

For this purpose, as shown in FIG. 7, the mitral annuloplasty band 2 in accordance with an exemplary embodiment of the present invention has a ratio between the width and height of 1:2 to 1:8, and preferably 1:4, as shown in FIG. 6, such that the band is transformable in a longitudinal direction (X direction) to bend or extend in a specific direction, and untransformable in a vertical direction (Z direction). That is, the band can lift up the mitral annulus to a certain height and fix it to the wall of the left atrium such that the band is transformable in the longitudinal direction, i.e., extends in a specific direction of the mitral annuloplasty band 2 depending on a contraction period of the heart, and is untransformable in the vertical direction to prevent disturbance of movement of the mitral annulus, the leaflet, and the left ventricle, thereby constantly maintaining the shape of the mitral annulus. Therefore, the ratio between the width and height is varied within a range of 1:2 to 1:8 depending on the material of the mitral annuloplasty band.

Moreover, as shown in FIG. 14, the annulus in the normal mitral valve is not located at the same level on an imaginary single plane (XY plane), and has a slightly varying height. Therefore, even when the annulus is bulged due to the mitral insufficiency, the height difference of the annulus is maintained.

As shown in FIGS. 14 to 16, the present invention can prevent bulge of the annulus and fix the annulus having varying height to fix the annulus located at the height of the mitral annuloplasty band 2 located on the left atrium to make the band 2 act as a reference plate. Since the mitral annuloplasty band 2 can prevent deformation of the annulus and provide probability for annuloplasty of the length and width of the leaflet and its subvalvular structure, the mitral annulus can be fixed to at least the height of the mitral annuloplasty band 2 in accordance with an exemplary embodiment of the present invention to prevent disturbance of movement of the mitral annulus and the left ventricle depending on contraction of the heart.

In a specific aspect, as shown in FIGS. 5 and 6, the mitral annuloplasty band 2 in accordance with an exemplary embodiment of the present invention may have a guide groove 4, especially a concave-shaped guide groove in a longitudinal direction, formed in a center of one surface of the rectangular parallelepiped in a longitudinal direction thereof.

Here, as shown in FIGS. 13 and 15, the guide groove 4 can make the rectangular parallelepiped-shaped mitral annuloplasty band 2 completely adhere to the wall of the left atrium and lift up the mitral annulus, and further securely stitch the mitral annulus lifted up to a certain height to the wall of the left atrium using a suture 6.

An adaptation of the mitral annuloplasty band in accordance with an exemplary embodiment of the present invention will be described below.

As shown in FIGS. 15 and 16, when mitral insufficiency occurs, after surgical correction of a disease of the mitral leaflet, the mitral annuloplasty band 2 is located so as to lift up the posterior mitral annulus to the sidewall of the left atrium adjacent to the mitral annulus and fix it thereto.

Here, in order to more readily describe an adaptation of the mitral annuloplasty band 2 in accordance with an exemplary embodiment of the present invention, as shown in FIGS. 8 and 9, after allocating the mitral annuloplasty band 2 at a position from C1 to C2 of the mitral valve, the band 2 and the mitral valve are stitched to each other using a suture.

Here, the dotted line of FIG. 8 shows a boundary for discriminating various sections of the mitral valve, for example, A1, A3, C2, P3, PM2, PM1, P1 and C1, which are typically designated by those skilled in the art. In addition, FIG. 9 shows positions where the mitral annuloplasty band 2 is stitched to the mitral valve when spread with respect to line B-B′ of FIG. 8.

Next, as shown in FIGS. 10 and 11, after stitching the mitral annuloplasty band 2 in place using the suture 6 and finishing the stitch, as shown in FIG. 12, the mitral annuloplasty band 2 lifts up the posterior mitral annulus to the sidewall of the left atrium and fixes it thereto.

While the mitral annuloplasty band 2 has a rectangular parallelepiped shape extending in a longitudinal direction thereof, the ratio of the width and height being in a range of 1:2 to 1:8, when the mitral annuloplasty band 2 having the guide groove 4 is used for a more effortless operation, the suture 6 can be stitched along the inner side of the mitral annuloplasty band 2, i.e., the guide groove 4.

Meanwhile, as shown in FIG. 13, the suture 6 in accordance with an exemplary embodiment of the present invention is used to stitch an edge of the mitral annulus from the wall of the left atrium without injury to the coronary arteries, and then, protrude through the mitral annuloplasty band 2, in particular, the guide groove 4 of the mitral annuloplasty band 2. In general, six to twelve double-armed sutures may be used.

When the mitral annuloplasty band 2 is stitched adjacent to the mitral valve of the left atrium through the abovementioned method, as shown in FIGS. 16 and 17, the posterior mitral annulus (shown as a thick solid line) adjacent to the posterior leaflet and loosened as shown in FIGS. 14 and 15 is lifted up to a certain height of the wall of the left atrium and fixed thereto.

While this invention has been described with reference to exemplary embodiments thereof, it will be clear to those of ordinary skill in the art to which the invention pertains that various modifications may be made to the described embodiments without departing from the spirit and scope of the invention as defined in the appended claims and their equivalents.

### INDUSTRIAL APPLICABILITY

In accordance with the present invention, a portion of the mitral annulus adjacent to the posterior leaflet can be lifted up to a mitral annuloplasty band located at the wall of the left atrium and fixed thereto to recover the function of the mitral valve, without disturbing movement of the mitral valve and movement of the mitral annulus and the left ventricle adjacent thereto.

1. A mitral annuloplasty band having a size that allows the band to be inserted into the atrium and a rectangular parallelepiped shape extending in a longitudinal direction thereof, wherein the ratio of the width to height is in a range of 1:2 to 1:8.

2. The mitral annuloplasty band according to claim 1, wherein a guide groove is longitudinally formed in a center of one side of the rectangular parallelepiped extending in the longitudinal direction.

3. The mitral annuloplasty band according to claim 1, wherein the mitral annuloplasty band is transformable in the longitudinal direction and untransformable in the vertical direction.

4. The mitral annuloplasty band according to claim 1, wherein the mitral annuloplasty band is formed of polytetrafluoroethylene, polypropylene, nylon, silk, polyurethane, polyester, or a mixture thereof.
